(19) United States
(12) Patent Application Publication
(10) Pub. No.: US 2011/0142848 A1
(43) Pub. Date: Jun. 16, 2011

(54) ANTI-BETA-2-MICROGLOBULIN AGENTS AND THE USE THEREOF

(76) Inventors: Leland W.K. Chung, Beverly Hills, CA (US); Sajni Josson, Decatur, GA (US); Wen-Chin Huang, Atlanta, GA (US); Haiyen E. Zhau, Beverly Hills, CA (US)

(21) Appl. No.: 13/056,926

(22) PCT Filed: Aug. 7, 2009
(86) PCT No.: PCT/US09/53091

§ 371 Date: Mar. 2, 2011

Related U.S. Application Data
(60) Provisional application No. 61/087,093, filed on Aug. 7, 2008.
(60) Provisional application No. 61/161,298, filed on Mar. 18, 2009.

(51) Int. Cl. A61K 39/395; A61K 31/7088; A61K 31/69; A61K 31/7068; A61K 33/24; A61K 31/704; A61K 31/337; A61K 31/7048; A61K 31/395; A61P 35/00; A61M 37/00

(52) U.S. Cl. 424/156.1; 424/174.1; 514/44 R; 514/64; 514/49; 424/649; 514/34; 514/449; 514/27; 514/183; 604/20

(57) ABSTRACT

A method for treating cancer includes treating a subject with an agent against beta-2-microglobulin; and treating the subject with radiation or a cancer therapeutic agent. The agent against beta-2-microglobulin includes anti-b2-M antibodies and miRNAs.

### CROSS-REFERENCE TO RELATED APPLICATIONS

This PCT application claims the priority of U.S. Provisional Patent Application No. 61/087,093, filed Aug. 7, 2008; and U.S. Provisional Patent Application No. 61/161,298, filed Mar. 18, 2009.

### BACKGROUND OF INVENTION

1. Field of the Invention

The invention relates generally to methods for treating cancers, and more particularly to methods for treating cancers with agents (e.g., antibodies) that inhibit beta2-microglobulin functions.

2. Background Art

β2-Microglobulin (β2-M) is a non-glycosylated protein composed of 119 amino acid residues, with a secreted form of 99 amino acids long that has a molecular mass of 11.8 KDa. β2-M is synthesized by all nucleated cells, and it can form a tertiary complex with the a chain of major histocompatibility complex (MHC) HLA class I antigens. Because β2-M is not firmly anchored on the cell membrane, it can be released from cells and exchanged with free extracellular β2-M protein.

β2-M has long been recognized as one of the most important factors contributing to host immunity. As cancer cells become immune-evasive, HLA class I antigens are lost, allowing cancer cells to escape the attacking cytotoxic T cells, and decrease steady-state levels of the cell-bound form of β2-M protein. However, in a number of liquid tumors (e.g., lymphomas, leukemia, and multiple myeloma) and solid tumors (e.g., prostate, breast, renal, and gastrointestinal), increased serum or urine β2-M has been reported. In some cases, serum β2-M levels may be correlated with poor prognosis unrelated to the patient's renal functions. In other cases, urine β2-M levels are correlated with poor survival in prostate cancer patients.

For example, β2-M protein expression may be directly regulated by androgen. The levels of β2-M expression in clinical specimens correlate with the prostate cancer Gleason score and the metastatic status in prostate cancer patients. In addition, β2-M may be a major growth factor and cell signaling molecule that promotes prostate cancer cell growth in culture and accelerates prostate tumor growth in mouse bone. In addition to human prostate cancer, β2-M is a major growth factor and signaling molecule in human renal cancer cells. These results are supported by the observations that: 1) β2-M is secreted by human androgen-sensitive LNCaP prostate cancer cells in response to androgen stimulation; 2) β2-M may be more specific than prostate-specific antigen (PSA), a well-established androgen-responsive target, as a marker for assessing androgen responsiveness; 3) serum and tissue β2-M levels correlated with the Gleason score and metastatic status of human prostate cancer; 4) in both human prostate and renal cancer cells, β2-M was shown to be a potent growth factor and signaling molecule and the overexpression of β2-M in these cells drives them to home in to bone in experimental models.

132-m may also support the growth of normal osteoblasts, rat and mouse stromal fibroblasts, and cancer cells by activation of some important signaling molecules, such as phosphoinositide 3-kinase (PI3K-Akt), mitogen activated protein kinase (MAPK), and cyclins. β2-M may activate survival by increasing the expression of survivin and androgen receptor. Furthermore, β2-M may promote angiogenesis through increasing the expression of VEGF and neuropilin (a VEGF receptor), reducing cell death signaling through decreased activation of caspases and JNK signaling activation, and increases bone turnover through activation of receptor activator of NF-κB ligand (RANKL).

The anti-β2-M mAbs may be used as therapeutic antibodies by specifically targeting the β2-M-mediated downstream pleiotropic signaling pathways. For example, the anti-β2-M mAbs may modulate the PI3K-AKT-, the MAPK-, the androgen receptor (AR), and the vascular endothelial growth factor (VEGF)-mediated survival signaling pathways in prostate and renal cancers. In addition, the anti-β2-M mAbs may block the growth and survival signaling pathways activated by growth factors and chemokines in multiple myelomas and leukemia.

The anti-β2-M mAbs treatment may not produce undesirable side effects based on the following observations: (1) immune competent mice with β2-M knockdown (equivalent biochemically to mice treated long-term with anti-β2-M mAbs) survived and gained weight as compared with the control animals that express intact β2-M. Some β2-M knockdown mice developed mild autoimmune syndromes, but overall β2-M knockdown did not affect significantly the life expectancy of the litter mates. (2) The anti-β2-M mAbs treatment results in a dramatic decrease in cancer cell growth without compromising the growth and survival of normal cells in vital organs. No acute toxicity or mortality is observed in immune-deficient mice treated with anti-β2-M mAbs. (3) The anti-β2-M mAbs also exerts a direct cytotoxic effect on cancer cells with no obvious toxicity against the normal cells, such as human blood cells, the lymphocytes implanted in immune compromised mice, and the cultured normal human prostate epithelial cells and fibroblasts.

β2-M protein may form a complex with MHC class I classical and non-classical proteins. β2-M may also determine the cell surface expression of these proteins. Upon malignant transformation, the expression of MHC classes I classical proteins may be decreased. The β2-M action may be mediated by the non-classical MHC-like proteins. Of interest is the non-classical MHC-like molecule, hemochromatosis gene (HFE) (FIG. 1). Hemachromatosis is an autosomal recessive disorder that may cause iron overload. As a result, it may lead to cirrhosis of the liver, diabetes, hypermelanotic pigmentation of the skin, heart failure, and liver cancer. One prominent feature associated with the many forms of this disorder is the HFE mutations. β2-M and HFE knockout mice and hemochromatosis patients have hypogonadotropic hypogonadism in some cases. Decrease in β2-M signaling in these cases causes regression of the prostate.

β2-M may interact with HFE and activate the downstream signaling pathways. Several lines of evidence support the notion that HFE (rather than MHC class 1 antigen) is a putative β2-M receptor. For example, (1) β2-M may promote the growth and activates the downstream signaling in prostate cancer cells that have undetectable level of MHC class 1 molecules. Moreover, the degree of inhibition induced by anti-β2-M mAbs in prostate cancer cells may be correlated inversely with cell surface expression of MHC-class I as determined by flow cytometry and Western blot. (2) Unlike MHC class I complex, which may be down regulated in malignant transformation, HFE expression remains steady in numerous normal cells and cancer cells. The differential sensitivity to the anti-β2-M mAbs-induced cytotoxicity between the cancer cells and the normal cells may be explained by the higher levels of transferrin receptor 1 (TFRC)— transferrin (TF) complex in cancer cells than that in the normal cells. (3) The anti-β2-M mAbs-induced prostate cancer cell death may be attenuated by an iron scavenger, desferrioxamine, suggesting that the anti-β2-M mAbs treatments may induce iron overload. (4) The β2-M/HFE complex may be co-immunoprecipitated using an anti-β2-M mAbs. (5) HFE-knockdown using lentiviral HFE sequence-specific siRNAs abolished the ability of anti-β2-M mAbs to induce prostate cancer cell death in vitro, suggesting that anti-β2-M mAbs-induced cell death requires HFE.

Furthermore, the β2-M/HFE complex is known to interact with transferrin receptor and functions as a negative regulator of iron uptake by inhibiting the TFRC function (FIG. 1). TFRC is the primary route of iron uptake in most cells. TFRC binds transferring (TF), which contains iron. TFRC/TF undergoes endosomal recycling and releases the iron into the cell. TF is the agonist for TFRC, whereas the β2-M/HFE complex is the antagonist for TFRC. Therefore, the absence of the 132-M/HFE complex, such as in hereditary hemochromatosis patients, may result in iron overload due to increased TFRC activity. Similarly, the anti-β2-M mAbs treatment may induce iron overload. It may be caused by inactivating the β2-M/HFE complex to serve as an antagonist for TFRC. As a result, iron overload may be induced in prostate cancer cells (FIG. 1). As expected, this effect can be reversed, in part, by an iron scavenger. Importantly, the anti-β2-M mAbs-induced cytotoxicity may be minimized in the normal cells because the normal cells express either low or undetectable levels of TFRC. In contrast, TFRC activity may be crucial for cancer cell growth, which may require abundant supply of iron for cell division. Thus, over-activating the TFRC in cancer cells by using anti-β2-M mAbs may cause excessive iron overload and reactive oxygen species (ROS) production, which may cause reduced DNA repair and increased DNA damage leading to apoptosis (FIG. 1). Therefore, anti-β2-M mAbs may be used to specifically target the cancer cells, and not the normal cells.

When the β2-M-mediated pleiotropic cell signaling pathways are interrupted by an anti-β2-M antibody (polyclonal or monoclonal antibody), the result may be a massive cell death in cancer cells, but not in normal cells. For example, in multiple myeloma (a bone cancer), anti-β2-M mAbs treatments may cause massive human and mouse myeloma cell death without affecting the normal cells. In addition, anti-β2-M antibody may induce apoptosis in both human prostate and renal cancer cells and in the established prostate tumors grown in mice by abrogating the survival signaling through, for example, down regulating a survival factor, androgen receptor (AR), and its target genes, e.g., prostate specific antigen (PSA) and survivin. In cells that do not express AR or PSA, anti-β2-M antibodies may cause oxidative stress, apoptosis and downregulated survival signaling in cancer cells. Together, these findings serve as a scientific basis for developing improved cancer treatment, e.g., treatment for human prostate and renal cancer bone and visceral organ metastases, in which the cancer cells may be specifically targeted and the side effects on the normal cells may be minimized.

### SUMMARY OF INVENTION

One aspect of the invention relates to a method for treating cancer. A method in accordance with one embodiment of the invention includes treating a subject with an agent against β2-M and treating the subject with radiation or a chemotherapeutic agent. The agent against β2-M may be an antibody (polyclonal or monoclonal) or an miRNA.

Another aspect of the invention relates to a kit for cancer therapy. A kit in accordance with one embodiment of the invention includes an agent against β2-M and a chemotherapeutic agent. The agent against β2-M may be an antibody (polyclonal or monoclonal) or an miRNA.

Other aspects and advantages of the invention will be apparent from the following description and the appended claims.

### BRIEF DESCRIPTION OF DRAWINGS

FIG. 1 is a schematic illustrating the potential mechanisms underlying some embodiments of the present invention.

FIG. 2 shows the effects on clongenic survival of prostate cancer cells after the treatments in accordance with one embodiment of the present invention.

FIG. 3 shows the effects on clongenic survival of prostate cancer cells after the treatments in accordance with another embodiment of the present invention.

FIG. 4 shows the effects on prostate tumor growth after the treatments in accordance with another embodiment of the present invention.

FIG. 5 shows the effects on protein expression after the treatments in accordance with another embodiment of the present invention.

FIG. 6 shows the effects on microRNA expression after the treatments in accordance with another embodiment of the present invention.

FIG. 7 shows the effects on proliferation of C4-2B (KD) cells after the treatments in accordance with one embodiment of the present invention.

FIG. 8 shows the effects on proliferation of C4-2B (KD) cells after the treatments in accordance with another embodiment of the present invention.

FIG. 9 shows the effects on proliferation of C4-2B (KD) cells after the treatments in accordance with another embodiment of the present invention.

FIG. 10 shows the effects on proliferation of C4-2B (KD) cells after the treatments in accordance with another embodiment of the present invention.

FIG. 11 shows the effects on proliferation of C4-2B (KD) cells after the treatments in accordance with another embodiment of the present invention.

FIG. 12 shows the effects on proliferation of PC-3 cells after the treatments in accordance with another embodiment of the present invention.

FIG. 13 shows the effects on proliferation of PC-3 cells after the treatments in accordance with another embodiment of the present invention.

FIG. 14 shows the effects on proliferation of PC-3 cells after the treatments in accordance with another embodiment of the present invention.

FIG. 15 shows the effects on proliferation of PC-3 cells after the treatments in accordance with another embodiment of the present invention.

FIG. 16 shows the effects on proliferation of PC-3 cells after the treatments in accordance with another embodiment of the present invention.

FIG. 17 shows the effects on proliferation of PC-3 cells after the treatments in accordance with another embodiment of the present invention.

FIG. 18 shows the effects on proliferation of PC-3 cells after the treatments in accordance with another embodiment of the present invention.

FIG. 19 shows the effects on proliferation of PC-3 cells after the treatments in accordance with another embodiment of the present invention.

FIG. 20 shows the effects on proliferation of PC-3 cells after the treatments in accordance with another embodiment of the present invention.

FIG. 21 shows the effects on proliferation of PC-3 cells after the treatments in accordance with another embodiment of the present invention.

FIG. 22 shows near infra red imaging of prostate tumors in wild type C57BL/6 mice, TRAMP mice treated with IgG antibody and TRAMP mice treated with anti-β2-M antibody in accordance with another embodiment of the present invention.

FIG. 23 shows the effects of antibody treatment on immune cell numbers in the spleen of immune competent, wild type C57BL/6 mice, TRAMP mice treated with IgG antibody and TRAMP mice treated with anti-β2-M antibody in accordance with another embodiment of the present invention.

### DETAILED DESCRIPTION

Embodiments of the invention relate to methods for treating cancers using an agent against β2-M. Some embodiments of the invention relate to methods for treating cancers with an agent against β2-M in combination with another modality for cancer treatment, such as radiation or chemotherapeutic agents. In such combination therapy, synergistic effects may be obtained due to sensitization of the cancer cells by the agent against β2-M, leading to more effective treatments and for lower toxicities.

Embodiments of the invention may be applied to any type of cancer and any type of anti-tumor agents (radiation or chemotherapeutics0. For clarity of illustration, the following description will mainly use prostate cancer and the anti-tumor agents, such as radiation and some common chemotherapeutic drugs used in the clinics. However, one of ordinary skill in the art would appreciate that the same approaches may be applied to other types of cancer, such as breast, lung, renal and osteosarcoma cancer, and a combination thereof; and other types of chemotherapeutic agents, such as therapeutic antibodies, small molecule drugs, nucleic acid-based drugs, and peptide-based drugs.

Embodiments in accordance with the present invention may include the use of anti-β2-M mAbs in combination with chemotherapeutic agents for the treatment of various cancers including prostate, breast, lung, renal, and osteosarcoma cancer, and a combination thereof. The chemotherapeutic agents, which may be combined with anti-β2-M antibodies, may include, but not limit to, PS-341, gemcitabine, cisplatin, doxorubicin, taxotere, VP-16 (Etoposide, Etopophos®, or Vepesid®), 17-AAG (17-demethoxygeldanamycin), etc. The specific activity of each of these chemotherapeutic agents is well known to a person of ordinary skill in the art. For example, taxotere can stabilize tubulin and, thus, inhibit mitosis. Gemcitabine can inhibit DNA elongation. Doxorubicin and VP-16 can inhibit the functions of topoisomerase II. Cisplatin can inhibit DNA synthesis by interchelating DNA. PS-341 (bortezomib, a proteasome inhibitor) can inhibit NF-KB activation and cell survival. 17-AAG can inhibit the function of heat shock protein 90 (HSP90). These chemotherapeutic agents are only used herein for the purpose of illustration. However, one skilled in the art would appreciate that other chemotherapeutic agents may also be combined with anti-β2-M antibodies for treating various types of cancer based on the embodiments of the invention.

Anti-β2-M mAbs sensitizes prostate cancer cells to the radiation-induced cell death in vitro and in vivo.

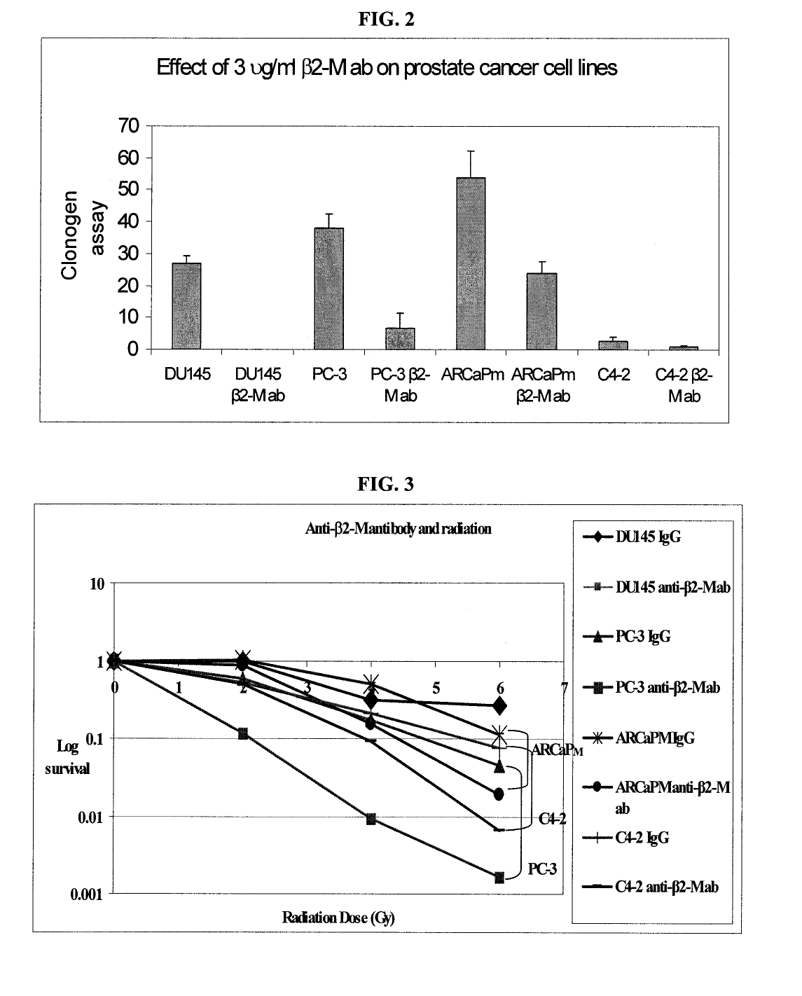

Anti-β2-M antibodies (polyclonal or monoclonal antibodies) have been found to inhibit tumor growth and are potentially therapeutic agents for various cancer treatments or prevention. For example, FIG. 2 shows the effects of anti-β2-M mAbs on prostate cancer cells (4 aggressive androgen-independent prostate cancer cell lines, e.g., ARCaP_{M}, C4-2, PC-3, and DU145) in a clongenic assay. In this study, cells were treated with 3 μg/ml of anti-β2-M mAbs for 48 h. The results show that ARCaP_{M} may be more resistant to anti-β2-M mAbs treatment than the other cell lines, whereas DU145 cells are more sensitive than the other cell lines. Nevertheless, anti-β2-M mAbs are effective against all these cancer cells.

From the results shown in FIG. 2, it is clear that anti-β2-M mAbs are effective against various cancer cells. In addition, the inventors have found that anti-β2-M mAbs can be used to enhanced the therapeutic efficacies of other cancer treatment modalities. For example, FIG. 3 shows that the Anti-β2-M mAbs treatment sensitizes ARCaP_{M}, PC-3 and C4-2 aggressive prostate cancer cells to the radiation-mediated cell killing.

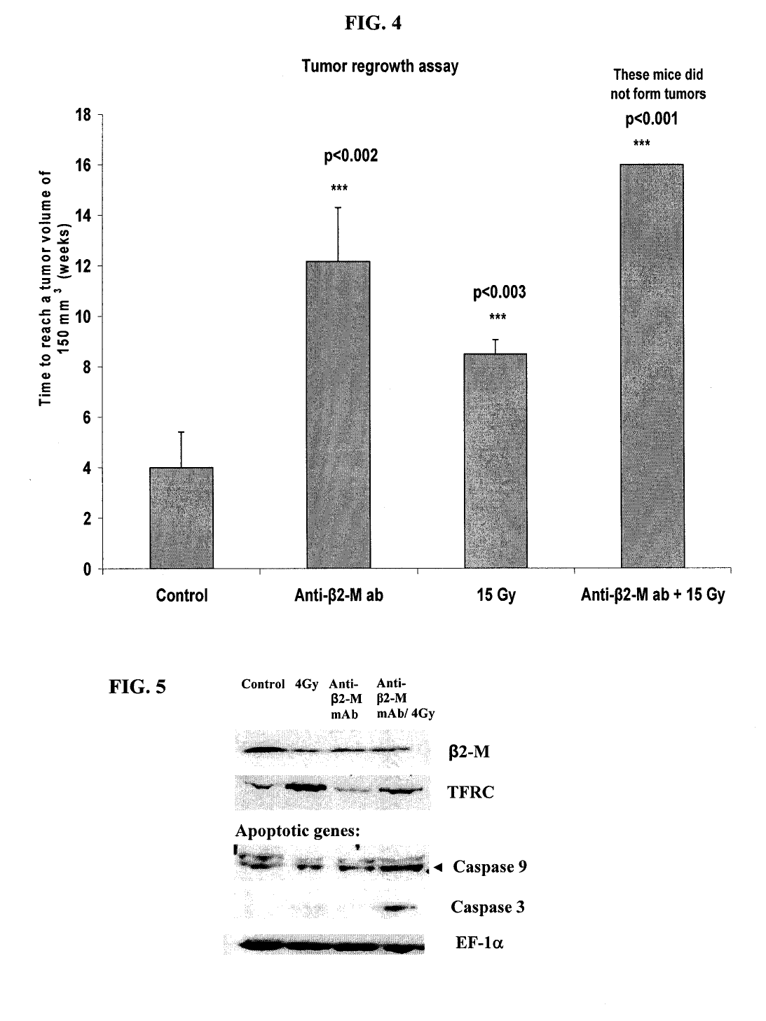

In the studies shown in FIG. 3, prostate cancer cells were treated with anti-β2-M mAbs (3 μg/ml) for 48 h, followed by ionizing radiation treatment at different doses. Clongenic assays were then performed after the radiation treatments. The results show that combined anti-β2-M mAbs and radiation treatments increase cell death by up to 100 folds in all aggressive prostate cancer cell lines. For example, DU-145 cells, which are known to be relatively radiation resistant, were rendered more sensitive to radiation (no cell survived) than other human prostate cancer cells. Similar radiation sensitization studies may be performed by combining radiation and anti-β2-M mAbs in vivo in an animal model, e.g., human prostate cancer xenografts using ARCaP_{M} tumors (see FIG. 4 below).

FIG. 4 shows that the combined treatment with a single injection of anti-β2-M mAbs (20 μg/ml) and radiation may inhibit tumor growth, as compared to the control treatments. Briefly, mice were subcutaneously injected with ARCaP_{M} cells on the flanks. When the tumors reach a size of 4 mm³, the tumors were treated with IgG or anti-β2-M mAbs, in Gelform®. The Gelform® may be immersed in the 20 μg/ml of antibodies and surgically implanted adjacent to the tumors. Twenty four hours later, tumors were irradiated at 15 Gy. Tumor volumes were subsequently monitored weekly. The tumor re-growth assay measures the time needed for a tumor to reach the volume of 150 mm³ after the initial treatment.

The results in FIG. 4 show that the control tumors re-grow rapidly (about 4 days), whereas the growth of the anti-β2-M mAbs and radiation treated tumor is significantly delayed (about 16 days). The anti-β2-M mAbs/radiation combination treatment prevents the growth at seven different tumor sites. Treatments with the anti-β2-M mAbs (about 12 days) or radiation (about 8 days) alone were not as effective. Importantly, the anti-β2-M mAbs treatment does not create toxic side effects on the mice. These results may be significant because the anti-β2-M mAbs may universally sensitize hormone refractory metastatic prostate cancer cells to radiation therapy. The anti-β2-M mAbs/radiation combined treatment, therefore, may represent a promising new modality for treating metastatic prostate cancer.

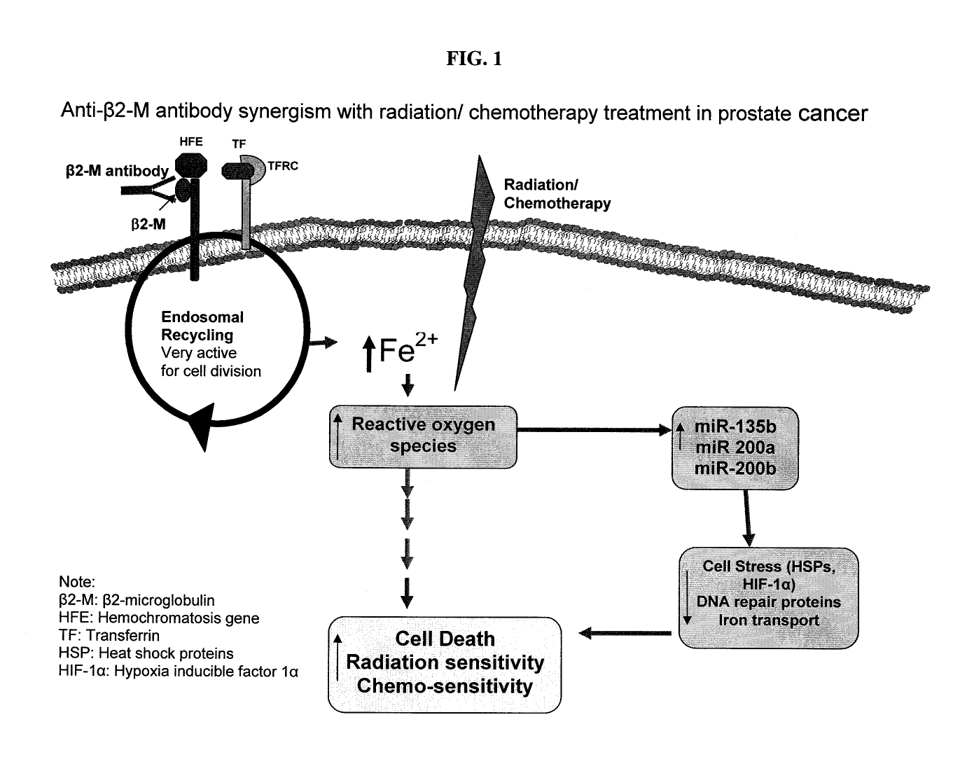

To understand the mechanisms of anti-β2-M mAbs actions, alone or in combination, the changes in various protein expressions were investigated. In particular, the expressions of TFRC, β2-M, and apoptotic proteins were found to be altered. For example, FIG. 5 shows that TFRC expressions were altered in response of anti-β2-M mAbs, radiation, and the combined anti-β2-M mAbs/radiation treatment in ARCaP_{M} prostate cancer organoids (3D model), which mimic tumors in vivo. In these studies, ARCaP_{M} cells were grown for 48 h in a 3D rotary wall vessel to form organoids. These organoids were treated with anti-β2-M mAbs (5 μg/ml) for 24 h followed by radiation with 4 Gy. Cells were lysed at 24 h after radiation, followed by Western blot analysis. The β2-M protein levels were decreased in all three treatments as compared to that of the control. While the anti-β2-M mAbs treatment decreased the levels of TFRC, the radiation treatment actually increased the levels of TFRC. Importantly, pre-treatment with the anti-β2-M mAbs blocked the induction of TFRC expression in response to radiation. These observations are consistent with the involvement of TFRC, as illustrated in FIG. 1.

Furthermore, certain apoptotic proteins, such as caspase 9 and 3, were significantly increased by the combination treatment, but not by separate anti-β2-M mAbs or radiation treatment. This result suggests that combination therapies using anti-β2-M mAbs and radiation (and other cancer treatment modality) induces tumor cell apoptosis.

In addition to the change of protein expressions, anti-β2-M mAbs also up-regulate miRNAs in ARCaP_{M} cells. MicroRNAs (miRNAs) may be post-transcriptional regulators of gene expression. By binding to mRNAs, these miRNAs may affect gene expression by inhibiting translation and/or increasing degradation of mRNAs. Each miRNA may target several mRNAs.

MiRNA screening may be performed by multiplexing quantitative real time PCR analysis. Using highly sensitive multiplexed, quantitative real-time PCR, 90 inducible miRNAs from prostate cancer cell lines can be tested. Briefly, a set of 90 miRNA may be monitored for changes in expression levels after the anti-β2-M mAbs treatment (3 μg/ml). For example, cells may be treated for 24 h followed by miRNA extraction. RNU6B may be used as internal control because its expression levels are not changed between groups. ARCaP_{E} cells are used as a control for being epithelial variant of ARCaP cells. Changes in miRNA expression levels may be presented as fold changes. The results show that the expression levels of certain miRNAs are changed.

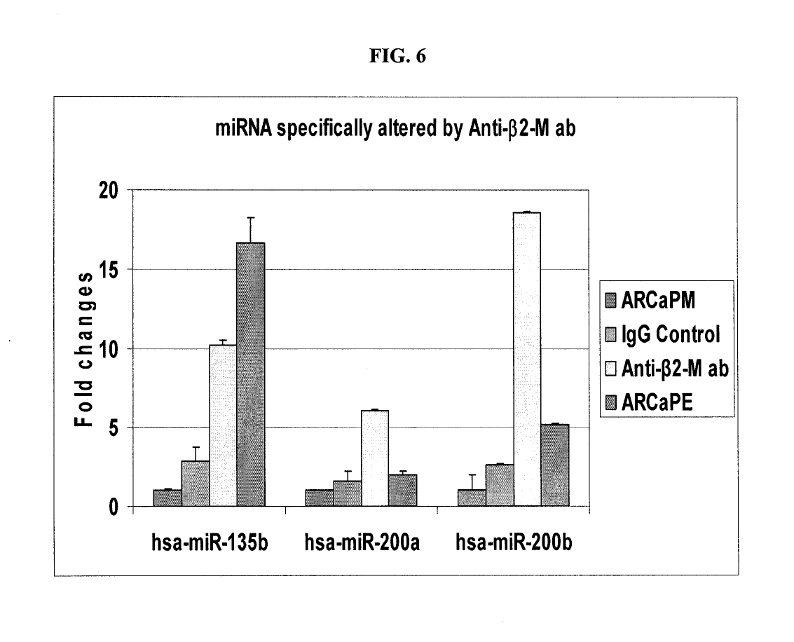

As shown in FIG. 6, the anti-β2-M mAbs treatment specifically increased the levels of miR-135b, miR-200a, and miR-200b. Upregulation of these miRNAs results in the downregulation of genes that control cell cycle and metastasis, such as hypoxia inducible factor-1α (HIF-1α), cyclinD2, pim2, phospholipase C γ1, TFRC, ZEB1 and ZEB2. Note that these anti-β2-M mAbs-induced miRNAs may function as β2-M antagonists similar to anti-β2-M mAbs. That is, these miRNAs themselves may be used as agents to counter the functions of β2-M.

The significance of miR-135b may be attributed to its ability to down-regulate oncogenes and growth/metastasis associated genes, e.g., HIF-1α, cyclin D2, pim-2, and phospholipase C γ1. Consistent with this view, C4-2 and C4-2B bone metastatic LNCaP human prostate cancer epithelial variants express 100-fold less miR-135b as compared to PrEC, a normal human prostate epithelial cell line. Thus, the anti-β2-M mAbs treatment may increase miR-135b levels, which, in turn, may decrease the endogenous levels of HIF-1α mRNA. Consistent with the findings that HIF-1α may be associated with radiation resistance, the HIF-1α suppressor, e.g., miR-135b, may be decreased 10-fold in C4-2 cells in response to radiation. In addition, an increase in HIF-1α expression may be observed in response to radiation in ARCaP_{M} 3-D organoids. Therefore, anti-β2-M mAbs may enhance radiation sensitization by up regulating miR-135b, which, in turn, may down regulate HIF-1α, rendering the cells sensitive to radiation. This may be one of the mechanisms by which anti-β2-M mAbs may synergize with radiation to promote cancer cell death.

The expression of miR-200 family members may be specifically induced by the anti-β2-M mAbs treatment in ARCaP_{M} prostate cancer cells. The miR-200 family members may also target TFRC. Other targets of miR-200 family members include genes controlling epithelial to mesenchymal transition (EMT) and metastasis, such as ZEB1, ZEB2, neuropilin, and others. As such, anti-β2-M mAbs may inhibit ZEB1 and ZEB2 by increasing the expression levels of miR-200. Reduction of E-cadherin in EMT may contribute to cell mobility and invasiveness. Because ZEB1 and ZEB2 function as repressors of E-cadherin, inhibition of ZEB1 and ZEB2 by miR-200 family members may, thus, restore E-cadherin. As a result, EMT may be suppressed and mesenchymal to epithelial transition (MET) promoted. Therefore, anti-β2-M mAbs may be used to selectively target prostate cancer cells by inhibiting EMT. miR-200 may also down regulate TFRC. The anti-β2-M mAbs treatment, therefore, may sensitize prostate cancer cells to radiation by both up regulating the miR-200 family and by down regulating TFRC, which may be associated with radiation resistance.

Because the opposite effects of anti-β2-M mAbs and radiation on TFRC (FIG. 5), the cancer killing may be maximized by proper scheduling for the delivery of both anti-β2-M mAbs and radiation based on the induction of TFRC and the iron-regulated redox signaling in the cells.

The above described show that a combination of anti-β2-M mAbs and radiation treatments can produce enhanced anti-tumor effects. The mechanisms involved in anti-β2-M mAb effects are clearly not dependent upon radiation or any particular anti-cancer treatment modality. Therefore, it is expected that the ability of anti-β2-M mAbs to sensitize cancer cells to other anti-cancer agents should equally apply to combination treatments with other anti-cancer agents, including chemotherapeutic agents. To show that this is true, the following describes experiments that demonstrate the synergistic effects of combined therapies using anti-β2-M mAbs and other chemotherapeutic agents. Specifically, the synergistic effects are shown using anti-β2-M mAbs and chemotherapeutic agents, as well as using chemotherapeutic agents in β2-M knockdown (KD) cells. β2-M knockdown mimics the effect of anti-β2-M mAbs.

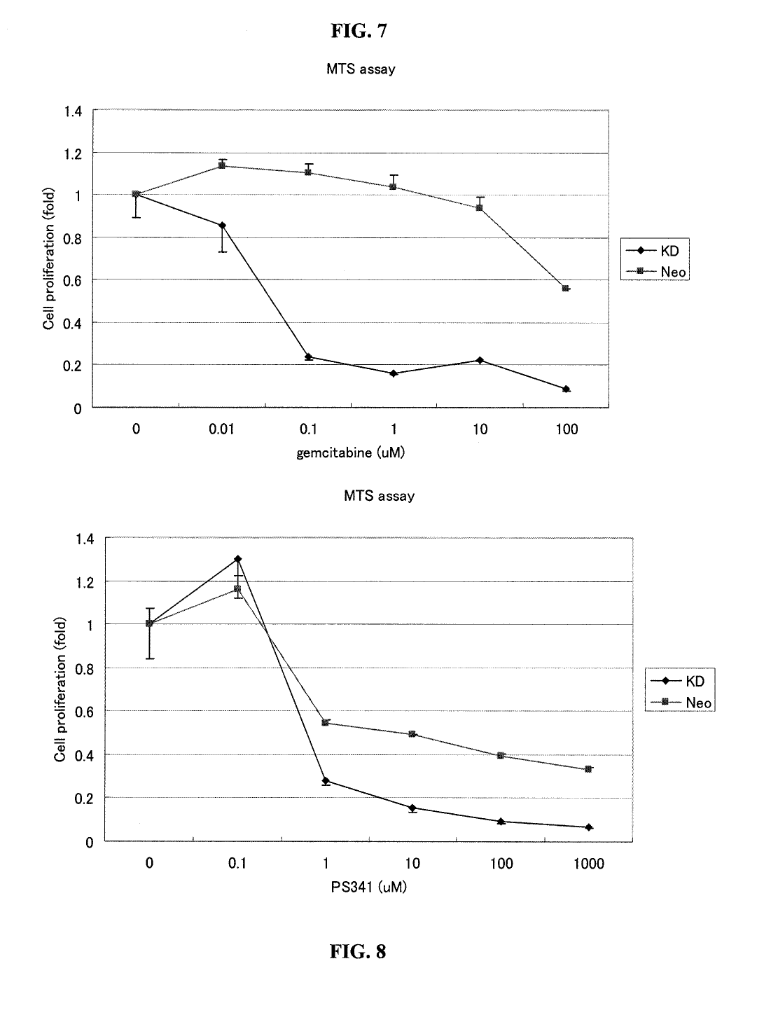

For example, an androgen-independent and androgen receptor-negative human prostate cancer cell line (PC-3) and a β2-M-knockdown androgen-independent cell line (C4-2B) may be used to demonstrate the synergism. For example, six thousand (6,000) cells per well may be plated in 96 well plates, which may be cultured in 1% DCC-FBS (dextran-coated, charcoal-treated fetal bovine serum) containing T medium (C4-2B) or serum-free T medium (PC3) with 5% CO₂ at 37° C. overnight. Cells may then be treated with anti-β2-M mAbs for 48 h. Chemotherapeutic agents, such as PS-341, gemcitabine, cisplatin, doxorubicin, taxotere, or 17-AAG, may then be added to the culture media. Two to four days post-treatment, cell growth may be determined by MTS assay using the CellTiter96 Aqueous One Solution Cell Proliferation Assay (Promega) according to the manufacturer's instructions. Results from these experiments are shown in FIGS. 7-21, with various chemotherapeutic agents.

In β2-M-knockdown mice, chemotherapeutic agents are found to have significantly enhanced effects, as compared to the effects in normal mice. For example, FIG. 7 shows that C4-2B (KD) (a β2-M-knockdown PC-3) cells are more sensitive to growth inhibition induced by gemcitabine, as compared with the neo-control cells. The results show that gemcitabine is not particularly effective a lower doses (10 μM or lower). However, β2-M-knockdown dramatically enhances gemcitabine's effects.

Similarly, FIG. 8 shows that C4-2B (KD) cells are more sensitive to cell growth inhibition induced by PS-341, as compared with the neo-control cells. Even though PS-341 is effective by itself at concentrations of 1 (AM or higher, β2-M-knockdown still produces an impressive enhancement.

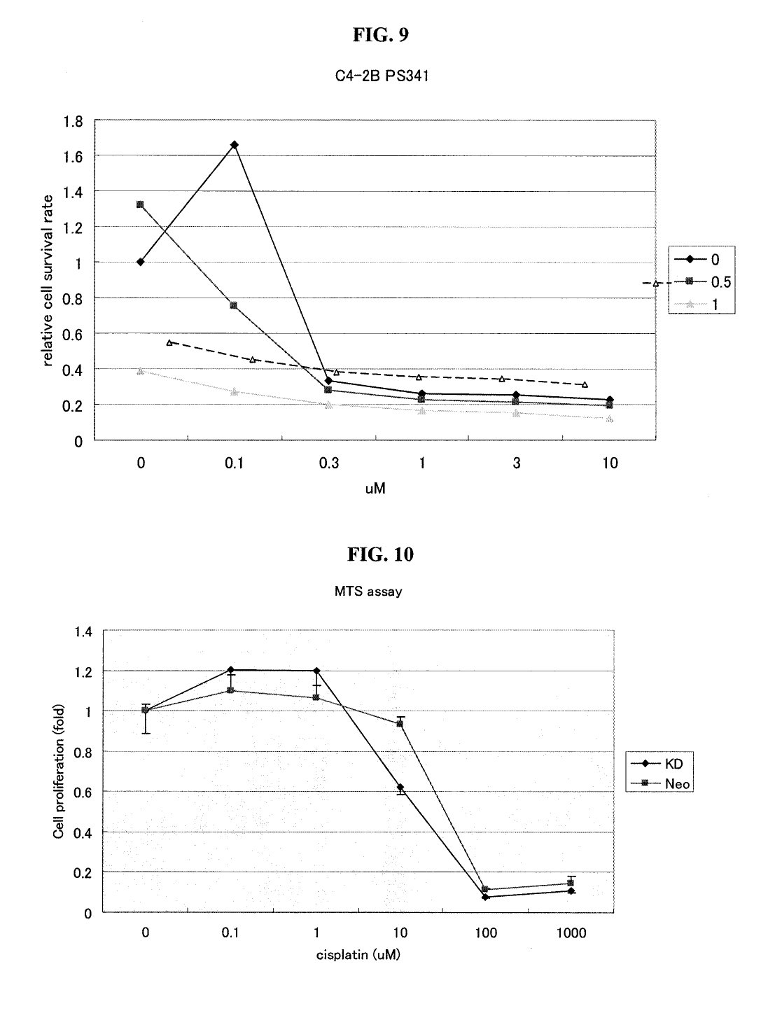

The enhancements are not only seen in β2-M-knockdown mice, but are also seen in normal mice treated with agents that antagonize or inhibit β2-M functions, such as antibodies against β2-M. For example, FIG. 9 shows that anti-β2M antibody (0.5, 1 μg/ml) can sensitize C4-2B (KD) cells to the cytotoxicity of PS-341. A comparison between the results shown in FIG. 8 and those in FIG. 9 confirms that the enhancements are indeed due to inhibition or knockdown of β2-M functions.

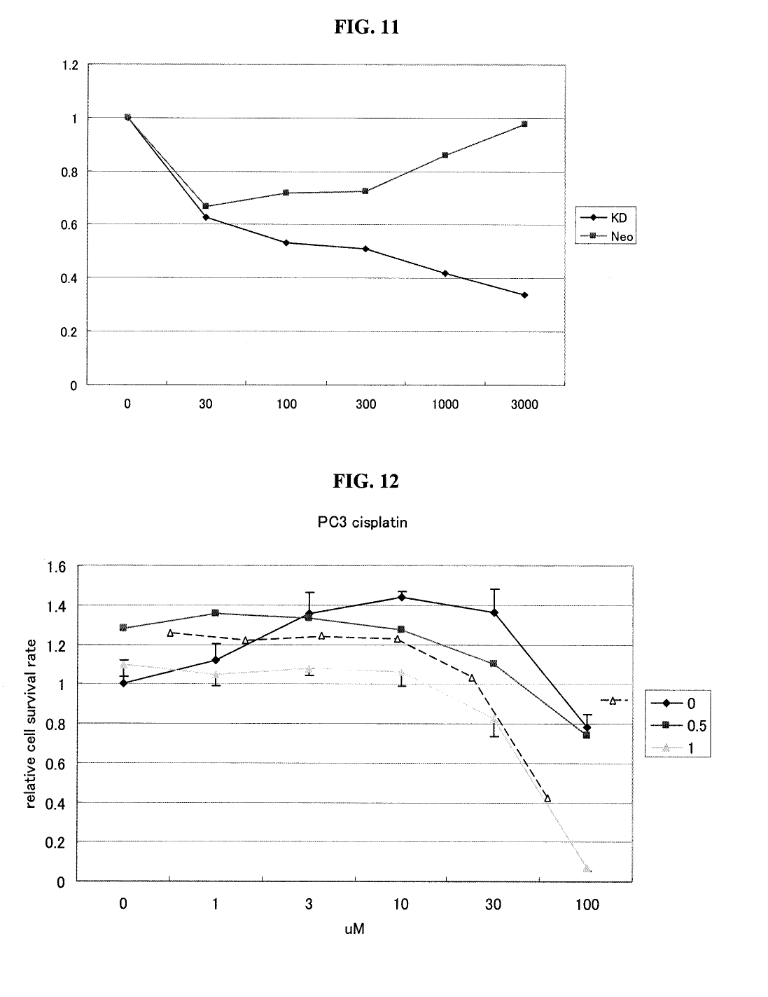

The enhancements are seen with various chemotherapeutic agents. For example, FIG. 10 shows that C4-2B (KD) cells are more sensitive to cell growth inhibition induced by cisplatin (e.g., 10 (AM), as compared with the neo-control cells. FIG. 11 shows that C4-2B (KD) cells are more sensitive to cell growth inhibition induced by taxotere than the neo-control cells.

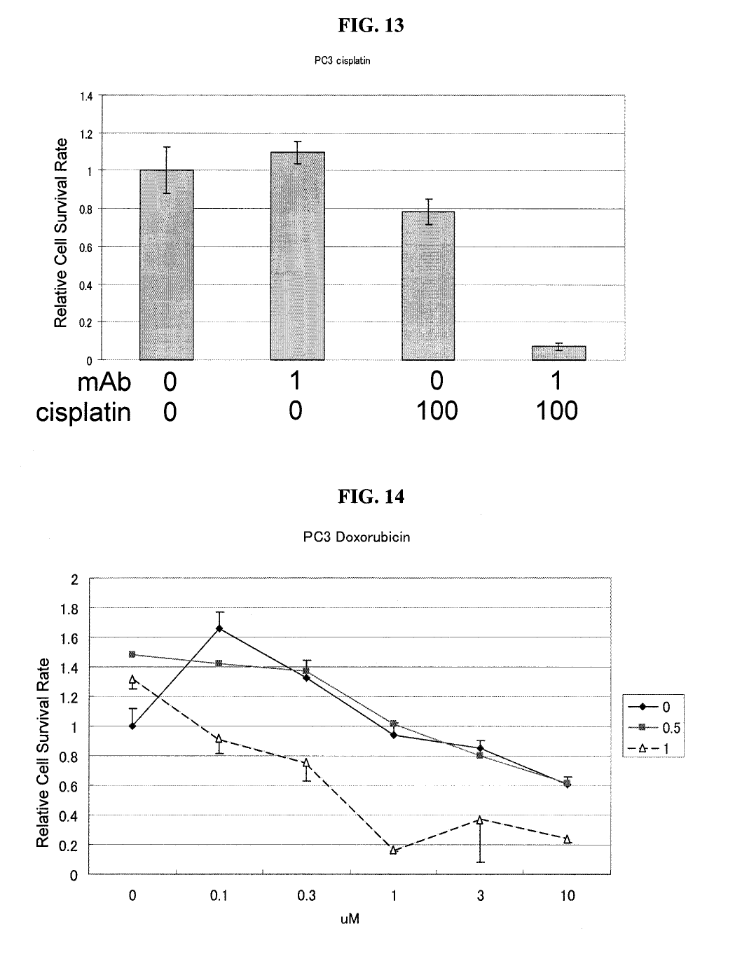

Furthermore, the enhancements are also seen with different cancer cells. FIG. 12 and FIG. 13 show that anti-β2M antibody treatment sensitizes PC-3 cells to the cytotoxicity of cisplatin. These results are similar to those with cisplatin in C4-2B (KD) cells.

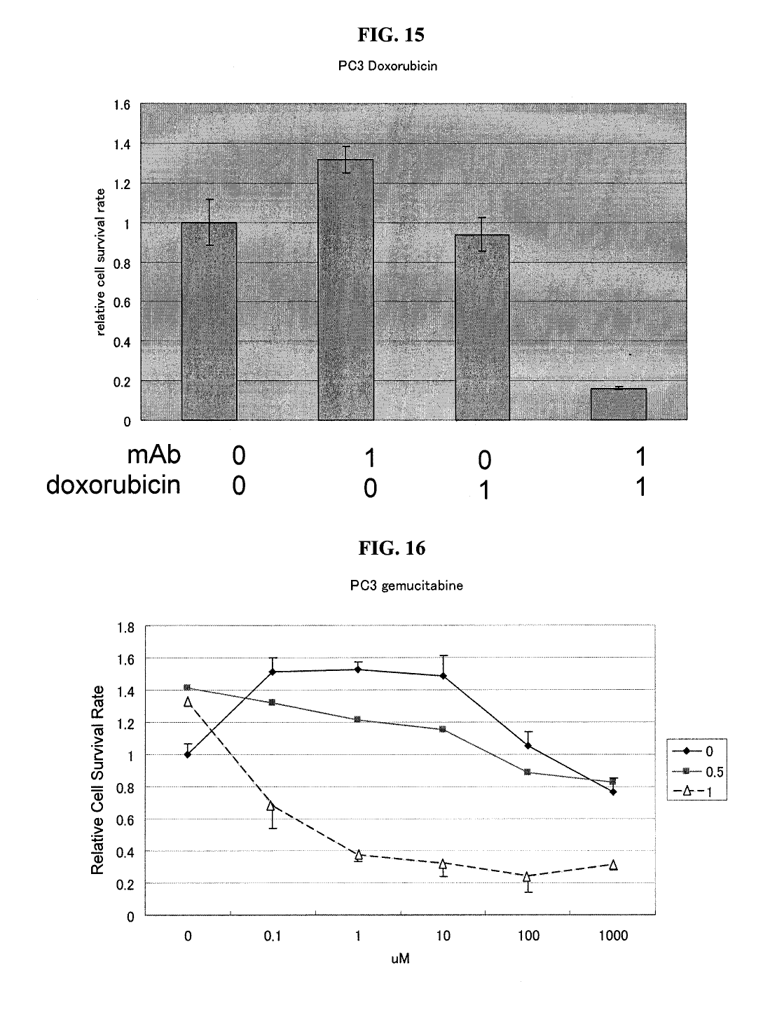

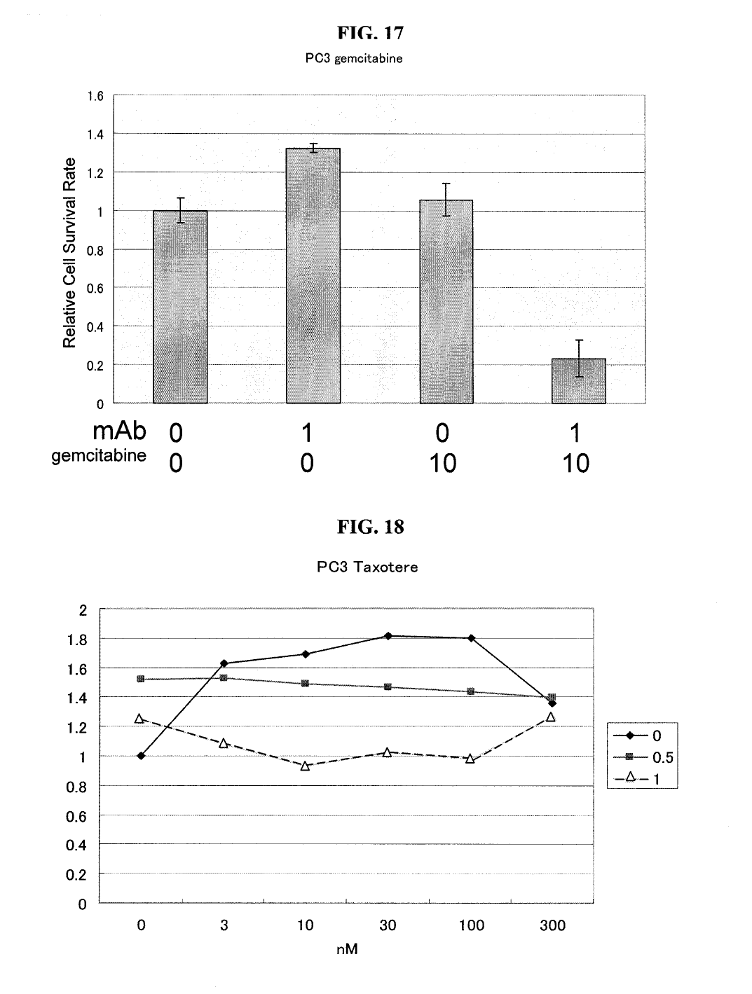

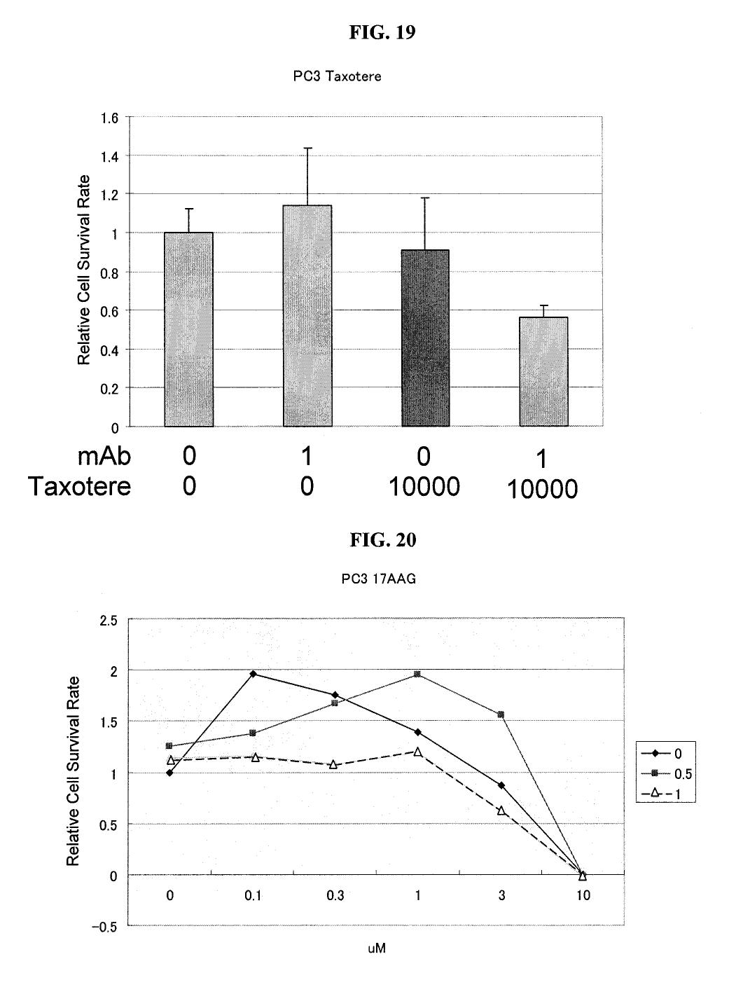

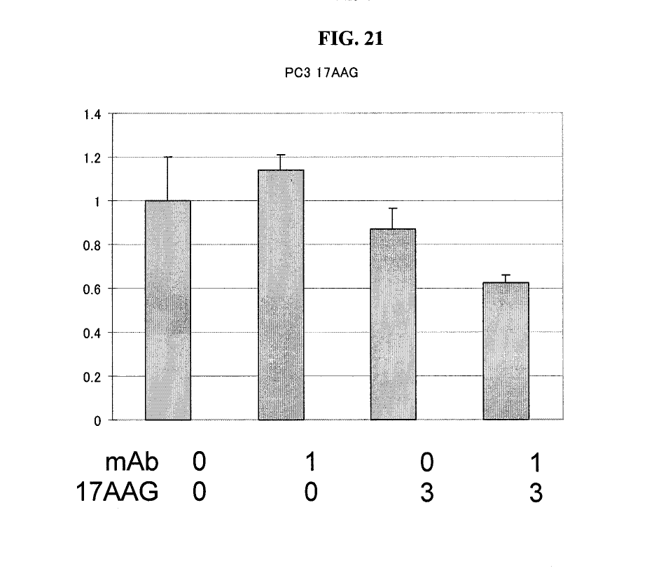

To further validate the general applications of anti-β2-M antibodies in enhancing the effects of other therapeutic agents, other chemotherapeutic agents are tested. FIG. 14 and FIG. 15 show that anti-β2M antibody treatment sensitizes PC-3 cells to the cytotoxicity of Doxorubicin. FIG. 16 and FIG. 17 show that anti-β2M antibody treatment sensitizes PC-3 cells to the cytotoxicity of gemcitabine. FIG. 18 and FIG. 19 show that anti-β2M antibody treatment sensitizes PC-3 cells to the cytotoxicity of taxotere. FIG. 20 and FIG. 21 show that anti-β2M antibody treatment sensitizes PC-3 cells to the cytotoxicity of 17-AAG.

Together, these results show that inhibition of β2-M functions contributes to sensitizing cancer cells to cancer therapy, either radiation therapy or chemotherapy. Therefore, any agents that can block or reduce β2-M functions (e.g., anti-β2-M mAbs or miRNAs described above) may be used to enhance the effects of other cancer treatment modalities, such as radiation or chemotherapeutic agents (e.g., PS-341, gemcitabine, cisplatin, doxorubicin, taxotere, and 17-AAG, etc).

Any antibodies against β2-M may be used, including monoclonal anti-β2M antibodies and polyclonal anti-β2M antibodies. For example, in the above in vitro studies and studies with nude mice experiments, monoclonal anti-β2M antibody from Santa Cruz Biotechnology (BBM.1) was used. For TRAMP mice studies, mAbs were generated using a hybridoma from ATCC (BBM.1), which was injected in mice to produce anti-β2-M monoclonal antibodies as ascites. The ascites were collected and purified using IgG purification kit and quantified before injection.

In addition to therapeutic effects, anti-β2-M antibodies are also found to have preventive effects. These preventive effects are demonstrated in TRAMP mouse model. The TRAMP model uses the rat androgen-dependent probasin gene promoter to drive the expression of simian virus 40 (SV40) large T and small t antigens in prostatic epithelium. Thus, the expression of SV40 antigens is specific to prostate and is hormonally and developmentally regulated. TRAMP mice will spontaneously develop prostate cancer.

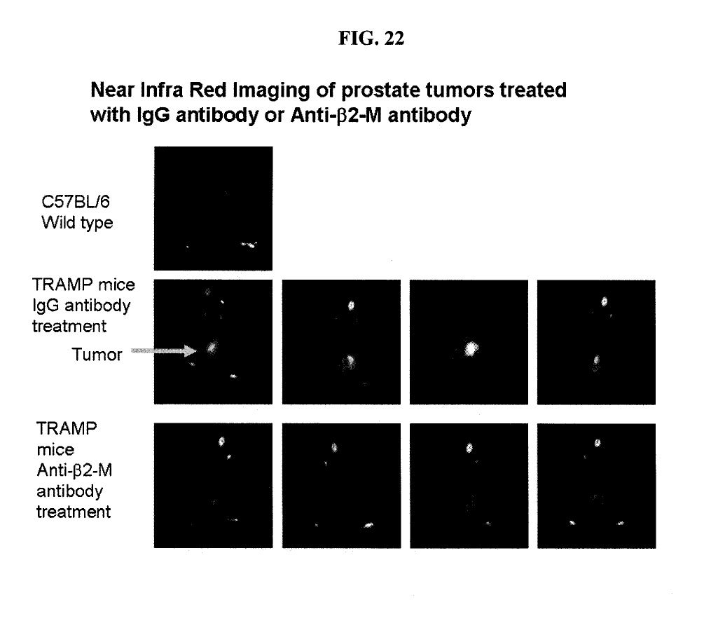

FIG. 22 shows tumor imaging studies in response to anti-β2-M mAbs in TRAMP mice. In this experiment, TRAMP mice and their background control C57BL/6 mice were used. T RAMP mice (n=4) were treated with either control IgG antibody or anti-β2-M mAbs. Mice were treated with 200 μg/ml purified mAbs on consecutive days, for two months, followed by two injections of 1 mg/ml mAbs on consecutive days. A week after treatments, mice were sacrificed. Imaging studies revealed that anti-β2-M mAb treatments prevented spontaneous development of prostate tumors in TRAMP mice, indicating that anti-β2-M antibodies can have preventive effects. These observations were confirmed by H/E staining.

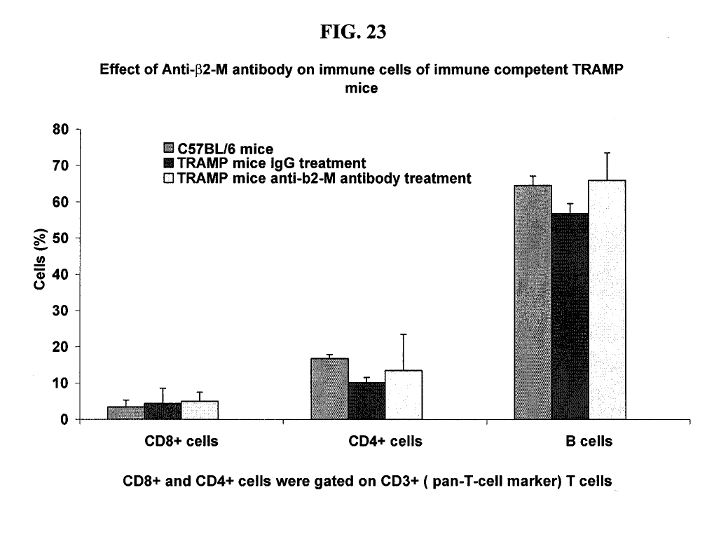

FIG. 23 shows immunological toxicity studies in response to anti-β2-M mAbs in TRAMP mice. Immune studies were performed on the spleens of these mice. Total T and B cell numbers were not affected in response to anti-β2-M mAb treatments, suggesting that anti-β2-M treatments would not interfere with host immune systems.

Advantages of embodiments of the invention may include one or more of the following. Methods of the invention may specifically sensitize cancer cells to radiation- and/or chemotherapeutic drug-induced cell killing without side effects affecting the normal cells. Therefore, methods of the invention may maximize treatment efficacy and, at the same time, minimize undesirable side effects.

Although embodiments of the invention are illustrated using a limited number of examples, one skilled in the art would appreciate that embodiments of the invention are not limited to these examples. Because β2-M is involved in a common mechanism that is not specific to any particular type of cancer, one skilled in the art would appreciate that embodiments of the invention can also be applies to other types of cancers. Furthermore, as illustrated above, anti-β2-M mAbs can render the cancer cells more sensitive to radiation and/or chemotherapeutic agents. Therefore, the synergistic effects are not expected to be specific to any particular chemotherapeutic agents. In other words, the synergistic effects illustrated above are generic and not limited to particular cancers or particular chemotherapeutic agents. Accordingly, the scope of the invention should be limited only by the attached claims.

1. A method for treating cancer, comprising:
treating a subject with an agent against beta-2-microglobulin; and
treating a subject with radiation or a cancer therapeutic agent.

2. The method of claim 1, wherein the agent against beta-2-microglobulin is an antibody.

3. The method of claim 2, wherein the antibody is a monoclonal antibody.

4. The method of claim 2, wherein the antibody is a polyclonal antibody.

5. The method of claim 1, wherein the agent against beta-2-microglobulin is an miRNA.

6. The method of claim 5, wherein the miRNA is at least one selected from the group consisting of miR-135, miR-200a, and miR-200b.

7. The method of claim 1, wherein the cancer is prostate cancer, breast cancer, lung cancer, renal cancer, osteosarcoma cancer, or a combination thereof.

8. The method of claim 1, wherein the chemotherapeutic agent is at least one selected from the group consisting of PS-341, gemcitabine, cisplatin, doxorubicin, taxotere, VP-16, and 17-AAG.

9. A kit for cancer therapy comprising:
an agent against beta-2-microglobulin; and
a chemotherapeutic agent.

10. The kit of claim 9, wherein the agent against beta-2-microglobulin is an antibody.

11-12. (canceled)
